(19) United States
(12) Patent Application Publication
(10) Pub. No.: US 2004/0014806 A1
(43) Pub. Date: Jan. 22, 2004

(54) Methods and compositions for lowering levels of blood lipids

(75) Inventors: B. Ganesh Bhat, St. Louis, MO (US); Devanand Luthria, St. Louis, MO (US); Anthony R. Torkelson, St. Louis, MO (US)

Correspondence Address: McDonnell Boehnen Hulbert & Berghoff, 32nd Floor, 300 S. Wacker Drive, Chicago, IL, 60606, US

(73) Assignee: Pharmacia Corporation

(21) Appl. No.: 10/385,033

(22) Filed: Mar. 10, 2003

Related U.S. Application Data
(60) Provisional application No. 60/362,871, filed on Mar. 8, 2002.

(51) Int. Cl. A61K 31/498; A61K 31/366; A61K 31/12

(52) U.S. Cl. 514/457; 514/249; 514/676; 514/680

(57) ABSTRACT

Disclosed are methods to lower blood cholesterol levels or inhibit ileal apical sodium co-dependent bile acid transport (ASBT) protein using coumarin and anthracene dione derivatives. Pharmaceutical compositions are also disclosed.

### BACKGROUND OF THE INVENTION

1. Field of the Invention

This invention relates to methods of using derivatives of coumarin and anthracene diones for inhibiting the ileal apical sodium co-dependent bile acid transport (ASBT) protein, and for lowering blood lipid levels. This invention also relates to pharmaceutical compositions comprising such compounds.

2. Description of the Related Art

Cholesterol is a biologically important molecule that can be obtained from dietary sources or synthesized by tissues, noteably the liver, in the human body. It is used for a multitude of purposes. All steroid hormones, including the mineralocorticoids and glucocorticoids, and the sex hormones, including progesterone, testosterone and estrogen, are made fromcholesterol. It is also used by the liver to synthesize bile acids and is a key component in the cell membranes of all cells.

High serum cholesterol levels are an important risk factor in the development of atherosclerosis and coronary artery disease as well, which is an extremely as other circulatory diseases and xanthomatosis. In addition, high serum cholesterol levels are seen in patients suffering from diseases including diabetes mellitus, familial hypercholesterolemia, acute intermittent prothyria, anorexia nervosa, nephrotic syndrome, primary cirrhosis and various liver disorders, such as hepatitis and obstructive jaundice.

As previously mentioned, the body converts cholesterol into bile acids, such as cholic acid and chemodeoxy-cholic acid, which are precursors to the conjugated salts of bile acids, such as taurocholate and glycocholate. Salts of bile acids act as detergents to solubilize lipids and consequently aid in digestion and absorption of dietary fats. Followingrelease into the small intestine, bile acids can be passively absorbed in the jejunum, or, in the case of conjugated bile acids, reabsorbed by active transport in the ileum. This reabsorption conserves cholesterol (in the form of bile acids) as it is taken up by the liver and recycled to the bile. Bile acids which are not reabsorbed in the gut are excreted. Therefore, reducing reabsorption of bile acids can diminish blood LDL cholesterol levels by stimulating the liver to use cholesterol for synthesis of more bile acids and causing an upregulation of the liver LDL receptors, which enhances clearance of LDL cholesterol thereby decreasing serum LDL cholesterol levels. See generally, Stedronsky, in “Interaction of bile acids and cholesterol with nonsystemic agents having hypocholesterolemic properties,” Biochimica et Biophysica Acta, 1210 (1994) 255-287; Reihnéer, E. et al, in “Regulation of hepatic cholesterol metabolism in humans: stimulatory effects of cholestyramine on HMG-CoA reductase activity and low density lipoprotein receptor expression in gallstone patients”, Journal of Lipid Research, Volume 31, 1990, 2219-2226; and Suckling et al, “Cholesterol Lowering and bile acid excretion in the hamster with cholestyramine treatment”, Atherosclerosis, 89(1991) 183-190.

In fact, reducing the reabsorption of bile acids has been recognized as a putative pharmaceutical target for the treatment of hypercholesterolemia. Kramer, et al, “Intestinal Bile Acid Absorption” The Journal of Biological Chemistry, Vol. 268, No. 24, Issue of August 25, pp. 18035-18046, 1993).

One method of reducing the amount of bile acids that are reabsorbed is oral administration of compounds that sequester (i.e. bond) the bile acids and cannot themselves be absorbed. The sequestered bile acids are consequently excreted.

Many bile acid sequestrants, however, do not bind bile acids well enough to prevent substantial portions from being reabsorbed. In addition, the volume of sequestrants that can be ingested with acceptable tolerability and safety is limited. As a result, the effectiveness of sequestrants to diminish blood cholesterol levels is also limited.

Another method of reducing the amount of bile acids that are reabsorbed is administering a compound that specifically inhibits or prevents the body's ability to reabsorb the bile acid. Blocking the ASBT protein with a specific inhibitor of this transporter will stimulate bile acid synthesis in the liver to replenish the bile acids lost due to increased excretion in the feces. This increased synthesis utilizes hepatic cholesterol as the precursor. As the hepatic cholesterol pool is depleted, it can be replenished by increasing de novo cholesterol synthesis (i.e., HMG CoA reductase activity) and by increasing LDL cholesterol uptake from the plasma (i.e. LDL receptor activity). The latter mechanism leads to a reduction in the blood LDL cholesterol level.

Compounds that interfere with the enterohepatic circulation system (i.e., reabsorption of bile acids in the intestine and back to the liver) are disclosed in Canadian Patent Application Nos. 2,025,294; 2,078,588; 2,085,782; and 2,085,830; and EP Application Nos. 0 379 161; 0 549 967; 0 559 064; and 0 563 731. In these applications, polymers of various naturally occurring constituents of the enterohepatic circulation system and their derivatives, including bile acids, are disclosed.

Clearly, inhibiting the reabsorption of bile acids to reduce blood LDL cholesterol levels is an important means to reduce cardiovascular risk. Thus, there is a need for efficacious and safe therapeutic agents that inhibit absorption of bile acids.

### SUMMARY OF THE INVENTION

The fruit and leaves of the tree Mammea Americana, a West Indian tree, contain coumarin and anthracene dione derivatives that inhibit bile acid reabsorption. These compounds can be obtained from natural sources or they can be prepared synthetically. They can be administered as individual therapeutic agents, as mixtures or with one or more pharmaceutically acceptable carriers, excipients, adjuvants and/or solvents. Thus, they may be administered as pharmaceutical compositions or “nutritional supplements.” Alternatively, they may be combined with or added to a patient's meal or food.

These compounds can also be coadministered with other therapeutic agents that are useful in lowering blood cholesterol levels, including the statin drugs, such as atorvastatin (Lipitor®), simvastatin (Zocor®), compactin, cerivastatin sodium tablets (Baycol®), pravastatin, rosuvastatin, lovastatin (Mevacor®), which prevent the body from synthesizing cholesterol, fibric acid derivatives such as gemfibrozil (Lopid®) and fenofibrate (Tricor®), and aspirin. Quite often, combination therapy exceeds the expected benefit from either therapy alone.

The coumarin and anthracene dione derivatives of the instant invention can also be administered with edible resins, and bile acid sequestrants that bind bile acids and prevents their reabsorption from the intestine. Examples include cholestyramine (Questran®), colestipol (Colestid®), and colesevelam HCl (WelChol™) Knapp, Howard et al., American Journal of Medicine, Apr. 1, 2001; 110:352-360. They could also be co-administered with agents that block cholesterol absorption from the intestine such as ezetimibe or an agent such as niacin that decreases tryglicerides and raises HDL levels.

This invention provides methods of using coumarin and anthracene dione compounds to lower blood cholesterol levels in a patient. In particular, the invention provides methods of inhibiting ASBT protein, which prevents the body from reabsorbing bile acids and causes the bile acids to be excreted; thereby causing the body to utilize circulating cholesterol in order to make more hepatic bile acids, and thereby reducing overall blood cholesterol levels.

The invention also provides for administering the compounds of the invention with other therapeutic agents and/or one or more pharmaceutically acceptable carriers, excipients or adjuvants.

The patient may be a human or other mammal. Treatment of humans, domesticated companion animals (pets) or livestock animals suffering from hypercholesterolemia with a therapeutically effective amount of a compound of the invention is contemplated by the invention.

One aspect of the invention is directed to a method of inhibiting the ileal bile acid transport protein (ASBT) by administering an effective amount of a compound of formula (I) and/or (II) to a mammal in need thereof:

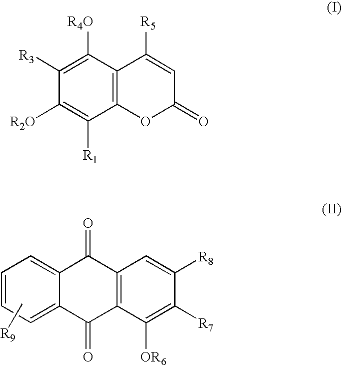

or their pharmaceutically acceptable salts,

wherein

R₁ and R₃ are independently hydrogen, alkyl, alkenyl, alkanoyl, —O-alkanoyl, arylalkanoyl, —O-arylalkanoyl, heteroarylalkanoyl, —O-heteroarylalkanoyl, or hydroxyalkyl, wherein each group is unsubstituted or substituted with 1, 2, or 3 groups that are independently alkyl, alkoxy, halogen, haloalkyl, haloalkoxy, nitro diazabicyclo[2.2.2]octyl;

R₂, R₄, and R₆ are independently hydrogen, alkyl, alkoxyalkyl, alkanoyl, aryl, arylalkanoyl, heteroarylalkanoyl,

wherein each group is unsubstituted or substituted with 1, 2, or 3 groups that are independently alkyl, alkoxy, halogen, haloalkyl, haloalkoxy, or diazabicyclo[2.2.2]octyl;

R₅ is hydrogen, alkyl, alkenyl, cycloalkyl, cycloalkylalkyl, aryl, arylalkyl, alkoxyalkyl, heteroaryl, heteroarylalkyl heterocycloalkyl, or heterocycloalkylalkyl,

wherein each group is unsubstituted or substituted with 1, 2, or 3 groups that are independently alkyl, alkoxy, halogen, haloalkyl, haloalkoxy, nitro, amino, diazabicyclo[2.2.2]octyl, mono or dialkylamino, carboxamido, or mono or dialkylcarboxamido;

R₇ and R₈ are independently alkyl, alkenyl, alkoxy, cycloalkyl, cycloalkylalkoxy, heterocycloalkyl, —CO₂H, —CO₂R₁₁,

wherein each of the above is optionally substituted with 1, 2, or 3 groups that are independently halogen, alkoxy, amino, diazabicyclo[2.2.2]octyl, or mono or dialkylamino;

wherein R₁₁ is alkyl, arylalkyl, aryl, or heterocycloalkylalkyl,

wherein each R₁₁ is optionally substituted with halogen, alkyl, alkoxy, hydroxy, haloalkyl, haloalkoxy, nitro, or diazabicyclo[2.2.2]octyl;

R₉ is selected from hydrogen, alkyl, alkoxy, halogen, CF₃, OCF₃, amino, mono or dialkylamino, carboxamido, or mono or dialkylcarboxamido,

wherein each alkyl group is optionally substituted with 1, 2, or 3 groups that are independently halogen, alkoxy, amino, diazabicyclo[2.2.2]octyl, or mono or dialkylamino.

Another aspect of the invention is directed to a method of lowering blood cholesterol levels comprising administering an effective amount of compounds of formulas (I) and/or (II).

Another aspect of the invention is directed to a pharmaceutical composition containing an effective amount of compounds of formulas (I) and/or (II).

### BRIEF DESCRIPTION OF THE DRAWINGS

FIG. 1 is a graph showing that taurocholate (a bile acid) uptake is dose-dependently inhibited by the leaf extract of mammea Americana.

FIG. 2 is a graph showing that the fruit extract of mammea Americana selectively inhibits the uptake of taurocholate compared to alanine.

FIG. 3 shows that the fruit extract of mammea Americana is not cytotoxic. In particular, FIG. 3 shows cell viability as determined by ATP (adenosine triphosphate) levels generated by living cells.

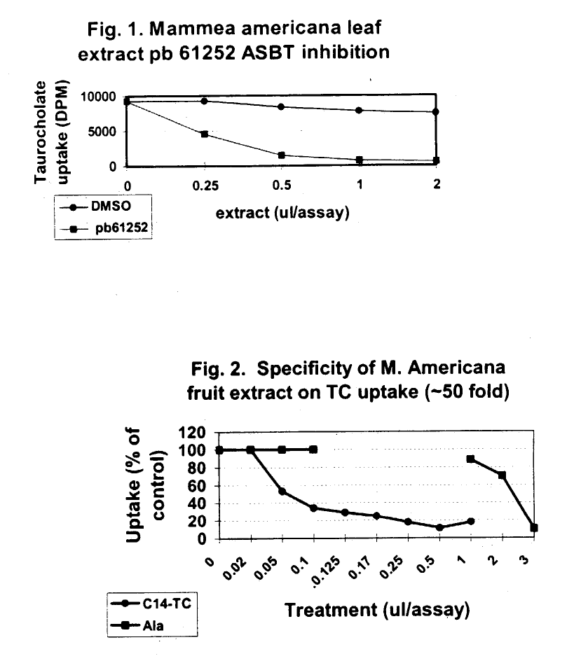

Further scope of the invention will become apparent from the detailed description provided below. However, it should be understood that the following detailed description and examples, while indicating preferred embodiments of the invention, are given by way of illustration only since various changes and modifications within the spirit and scope of the invention will become apparent to those skilled in the art from this detailed description.

### DETAILED DESCRIPTION OF THE INVENTION

In one specific embodiment, the method of inhibiting uptake of bile acid salts, for example, taurocholate, by ASBT protein comprises administering an effective amount of compounds (I) and/or (II) wherein R₅ is phenyl. In another specific embodiment, R₅ is phenyl and R₂ and R₄ are independently hydrogen, C₁-C₈ alkyl or alkanoyl. In yet another embodiment, R₅ is phenyl, R₂ and R₄ are independently hydrogen, C₁-C₈ alkyl or alkanoyl, and R₁ and R₃ are independently hydrogen, C₁-C₈ alkyl or alkanoyl.

In a second specific embodiment, the method of inhibiting uptake of bile acid salts, preferably taurocholate, by ASBT protein comprises administering an effective amount of compounds (I) and/or (II) wherein R₉ is hydrogen. In another embodiment, R₉ is hydrogen and R₇ is alkyl, alkenyl or alkanoyl. In yet another embodiment, R₉ is hydrogen and R₇ is alkyl, alkenyl or alkanoyl, and R₈ is hydrogen, alkyl, alkoxy or heterocycloalkyl, each of which is optionally substituted with up to four groups independently selected from C₁-C₆ alkyl, C₁-C₆ alkoxy, and halogen.

In a preferred embodiment, the method of inhibiting uptake of a bile acid salt such as taurocholate by ASBT protein comprises administering an effective amount of compound (I).

In another preferred embodiment, the method of inhibiting uptake of a bile acid salt such as taurocholate by ASBT protein comprises administering an effective amount of compound (II).

Another specific embodiment of the invention is directed to a method of inhibiting uptake of bile acid salts, preferably taurocholate, by ASBT protein comprising administering an effective amount of at least one of:

5,7-Dihydroxy-8-(3-methyl-but-2-enyl)-6-(2-methyl-butyryl)-4-phenyl-chromen-2-one;

5,7-Dihydroxy-6-(3-methyl-but-2-enyl)-8-(3-methyl-butyryl)-4-phenyl-chromen-2-one;

5,7-Dihydroxy-6-(3-methyl-but-2-enyl)-8-(2-methyl-butyryl)-4-phenyl-chromen-2-one;

5,7-Dihydroxy-8-(3-methyl-but-2-enyl)-6-(3-methyl-butyryl)-4-phenyl-chromen-2-one; or

1-Hydroxy-2-(2-methyl-allyl)-3-(4,4,6-trimethyl-[1,3]dioxan-2-yl)-anthraquinone.

In a preferred aspect, these compounds are administered together with a pharmaceutically acceptable carrier, excipient, adjuvant or solvent.

In another embodiment, the method of lowering blood cholesterol levels comprises administering an effective amount of compounds of formulas (I) and/or (II) to a mammal in need thereof, wherein R₅ is phenyl. In still another embodiment, R₅ is phenyl and R₂ and R₄ are independently hydrogen, C₁-C₈ alkyl or alkanoyl. In yet another specific embodiment, R₅ is phenyl, R₂ and R₄ are independently hydrogen, C₁-C₈ alkyl or alkanoyl, and R₁ and R₃ are independently hydrogen, C₁-C₈ alkyl or alkanoyl.

In another aspect, the method of lowering blood cholesterol levels comprises administering an effective amount of compounds of formulas (I) and/or (II) to a mammal in need thereof wherein R₉ is hydrogen. In a more preferred aspect, R₉ is hydrogen and R₇ is alkyl, alkenyl or alkanoyl.

In preferred embodiment, the method of lowering blood cholesterol levels comprising administering an effective amount of a compound of formulas (I).

In another preferred embodiment, the method of lowering blood cholesterol levels comprising administering an effective amount of a compound of formulas (II).

Another preferred embodiment of the invention is directed to a method of lowering blood cholesterol levels comprising administering an effective amount of at least one of:

5,7-Dihydroxy-8-(3-methyl-but-2-enyl)-6-(2-methyl-butyryl)-4-phenyl-chromen-2-one;

5,7-Dihydroxy-6-(3-methyl-but-2-enyl)-8-(3-methyl-butyryl)-4-phenyl-chromen-2-one;

5,7-Dihydroxy-6-(3-methyl-but-2-enyl)-8-(2-methyl-butyryl)-4-phenyl-chromen-2-one;

5,7-Dihydroxy-8-(3-methyl-but-2-enyl)-6-(3-methyl-butyryl)-4-phenyl-chromen-2-one; or

1-Hydroxy-2-(2-methyl-allyl)-3-(4,4,6-trimethyl-[1,3]dioxan-2-yl)-anthraquinone; and an optionally included carrier, excipient, adjuvant or solvent.

Preferred pharmaceutical compositions contain an effective amount of compounds of formulas (I) and/or (II) wherein R₅ is phenyl. More preferably, R₅ is phenyl, and R₂ and R₄ are independently hydrogen, C₁-C₈ alkyl or alkanoyl. Even more preferably, R₅ is phenyl, R₂ and R₄ are independently hydrogen, C₁-C₈ alkyl or alkanoyl, and R₁ and R₃ are independently hydrogen, C₁-C₈ alkyl or alkanoyl.

Other preferred pharmaceutical compositions contain an effective amount of compounds of formulas (I) and/or (II) wherein R₉ is hydrogen. More preferably, R₉ is hydrogen and R₇ is alkyl, alkenyl or alkanoyl. Still more preferably, R₉ is hydrogen, R₇ is alkyl, alkenyl or alkanoyl, and R₈ is hydrogen, alkyl, alkoxy or heterocycloalkyl, each of which is optionally substituted with up to four groups independently selected from C₁-C₆ alkyl, C₁-C₆ alkoxy, and halogen.

In another preferred embodiment, the invention provides pharmaceutical compositions containing an effective amount of at least one compound of formula (I).

In another preferred embodiment, the invention provides pharmaceutical compositions containing an effective amount of at least one compound of formula (II).

In another aspect, the invention provides pharmaceutical compositions for the prophylaxis or treatment of a disease or condition for which a bile acid transport inhibitor is indicated, such as a hyperlipidemic condition, for example, atherosclerosis. Such compositions comprise any of the compounds disclosed above, alone or in combination, in an amount effective to reduce bile acid levels in the blood, or to reduce transport thereof across digestive system membranes, and a pharmaceutically acceptable carrier, adjuvant, excipient, or diluent.

The compounds of this invention may contain one or more asymmetric carbon atoms, so that the compounds can exist in different stereoisomeric forms. These compounds can be, for example, racemates, chiral non-racemic or diastereomers. In these situations, the single enantiomers, i.e., optically active forms can be obtained by asymmetric synthesis or by resolution of the racemates. Resolution of the racemates can be accomplished, for example, by conventional methods such as crystallization in the presence of a resolving agent; chromatography, using, for example a chiral HPLC column; or derivatizing the racemic mixture with a resolving reagent to generate diastereomers, separating the diastereomers via chromatography, and removing the resolving agent to generate the original compound in enantiomerically enriched form. Any of the above procedures can be repeated to increase the enantiomeric purity of a compound.

When the compounds described herein contain olefinic double bonds or other centers of geometric asymmetry, and unless otherwise specified, it is intended that the invention include the cis, trans, Z- and E-configurations. Likewise, all tautomeric forms of the compounds are also intended to be included.

As used herein, the term “alkanoyl” refers to a straight or branched chain hydrocarbon attached to the parent molecular moiety through a carbonyl group. The hydrocarbon contains from 1-7 carbon atoms, preferably 1-5 carbon atoms, and more preferably 1-3 carbon atoms.

As used herein, the term “alkenyl” refers to a straight or branched hydrocarbon containing at least one carbon-carbon double bond. Examples of “alkenyl” include vinyl, allyl, and 2-methyl-3-heptene. Alkenyl groups herein contain from 2-8 carbon atoms, preferably from 2-6 carbon atoms and one carbon-carbon double bond.

As used herein, the term “alkyl” includes straight or branched saturated hydrocarbons. Alkyl groups herein contain at least one and no more than eight carbon atoms. Preferred alkyl groups contain 1 to 6 carbon atoms; more preferred alkyl groups have from 1 to 4 carbon atoms, while particularly preferred alkyl groups have from 1 to 3 carbon atoms. Examples of “alkyl” include methyl, ethyl, propyl, isopropyl, butyl, iso-, sec- and tert-butyl, pentyl, hexyl, heptyl, 3-ethylbutyl, and the like.

As used herein, the term “alkoxy” represents an alkyl group attached to the parent molecular moiety through an oxygen bridge. Examples of alkoxy groups include, for example, methoxy, ethoxy, propoxy and isopropoxy.

The term “aryl” refers to a hydrocarbon ring system containing at least one aromatic ring. The aromatic ring may optionally be fused or otherwise attached to other aromatic hydrocarbon rings or non-aromatic hydrocarbon rings. Examples of aryl groups include, for example, phenyl, naphthyl, 1,2,3,4-tetrahydronaphthalene and biphenyl. Preferred examples of aryl groups include phenyl and naphthyl. The aryl groups are unsubstituted, e.g., unsubstituted phenyl, or are substituted in any substitutable position with groups such as, for example, alkyl, alkoxy, alkenyl, halogen, haloalkyl, haloalkoxy, nitro, amino, mono- or dialkylamino, aminoalkyl, heterocycloalkyl, cycloalkyl, cycloalkylalkyl, etc. Preferred aryl substituents are C₁-C₄ alkyl, C₁-C₄alkoxy, C₁-C₄ alkenyl, halogen, halo (C₁-C₄)alkyl, nitro, amino, and mono- or di(C₁-C₃)alkylamino. Another preferred aryl subsittuent is diazabicyclo[2.2.2]octyl. Aryl groups such as phenyl and naphthyl can be substituted with up to 5, more preferably 3, most preferably 1 or 2, of the groups listed above.

The term “—O—” refers to a divalent oxygen-linking group.

The term “a bile acid transport inhibitor” means a compound capable of inhibiting absorption of bile acids from the intestine into the circulatory system of a mammal, such as a human. Such compounds are capable of increasing the fecal excretion of bile acids, as well as reducing the blood plasma or serum concentrations of cholesterol and cholesterol ester, and more specifically, reducing LDL and VLDL cholesterol. Conditions or diseases that benefit from the prophylaxis or treatment by bile acid transport inhibition include, for example, a hyperlipidemic condition such as atherosclerosis.

The term “cycloalkyl” refers to a C₃-C₈ cyclic hydrocarbon. Examples of cycloalkyl include cyclopropyl, cyclobutyl, cyclopentyl, cyclohexyl, cycloheptyl and cyclooctyl.

The term “cycloalkylalkyl,” as used herein, refers to a C₃-C₈ cycloalkyl group attached to the parent molecular moiety through an alkyl group, as defined above. Examples of cycloalkylalkyl groups include cyclopropylmethyl and cyclopentylethyl.

The terms “halogen” or “halo” indicate fluorine, chlorine, bromine, or iodine.

“Haloalkyl” refers to radicals wherein any one or more of the alkyl carbon atoms is substituted with halogen as defined above. Specifically embraced are monohaloalkyl, dihaloalkyl and polyhaloalkyl radicals. A monohaloalkyl radical, for one example, may have either an iodo, bromo, chloro or fluoro atom within the radical. Dihalo and polyhaloalkyl radicals may have two or more of the same halo atoms or a combination of different halo radicals. “Lower haloalkyl” embraces radicals having 1-6 carbon atoms. Examples of haloalkyl radicals include fluoromethyl, difluoromethyl, trifluoromethyl, chloromethyl, dichloromethyl, trichloromethyl, trichloromethyl, pentafluoroethyl, heptafluoropropyl, difluorochloromethyl, dichlorofluoromethyl, difluoroethyl, difluoropropyl, dichloroethyl and dichloropropyl.

The term haloalkoxy, as used herein, refers to haloalkyl as defined above connected to a parent group, e.g., aryl or heteroaryl, by an oxygen linker.

The term “heteroaryl” refers to an aromatic ring system containing at least one heteroatom selected from nitrogen, oxygen, and sulfur. The heteroaryl ring may be fused or otherwise attached to one or more heteroaryl rings, aromatic or non-aromatic hydrocarbon rings or heterocycloalkyl rings. Examples of heteroaryl groups include, for example, pyridyl, furanyl, thiophenyl, 5,6,7,8-tetrahydroisoquinolinyl and pyrimidinyl. Preferred heteroaryl groups include thienyl, benzothienyl, pyridyl, quinolyl, pyrazinyl, pyrimidinyl, imidazolyl, benzimidazolyl, furanyl, benzofuranyl, thiazolyl, benzothiazolyl, isoxazolyl, oxadiazolyl, isothiazolyl, benzisothiazolyl, triazolyl, tetrazolyl, pyrrolyl, indolyl, pyrazolyl, and benzopyrazolyl. The heteroaryl groups are unsubstituted or are substituted with for example, alkyl, alkoxy, alkenyl, halogen, haloalkyl, haloalkoxy, nitro, amino, mono- or dialkylamino, aminoalkyl, heterocycloalkyl, cycloalkyl, cycloalkylalkyl, etc. Preferred aryl substituents are C₁-C₄ alkyl, C₁-C₄alkoxy, C₁-C₄ alkenyl, halogen, halo (C₁-C₄)alkyl, nitro, amino, and mono- or di(C₁-C₃)alkylamino. Another preferred heteroaryl substituent is diazabicyclo[2.2.2]octyl. Aryl groups such as phenyl and naphthyl can be substituted with up to 5, more preferably 3, most preferably 1 or 2, of the groups listed above.

Highly preferred heteroaryl groups as pyridyl, imidazolyl, and pyrimidinyl.

The term “heterocycloalkyl,” refers to a non-aromatic ring system containing at least one hetero atom selected from nitrogen, oxygen, and sulfur. The heterocycloalkyl ring may be optionally fused to or otherwise attached to other heterocycloalkyl rings and/or non-aromatic hydrocarbon rings. Preferred heterocycloalkyl groups have from 3 to 7 members. Examples of heterocycloalkyl groups include, for example, piperazinyl, morpholinyl, piperidinyl, tetrahydrofuranyl, pyrrolidinyl, and pyrazolyl. Preferred heterocycloalkyl groups include piperidinyl, piperazinyl, pyrolidinyl, dioxolanyl, and dioxanyl.

The term “—O-arylalkanoyl” refers to an alkanoyl group attached to an aryl group, which is attached to the parent molecular moiety through an oxygen atom.

The term “—O-heteroarylalkanoyl” refers to an alkanoyl group attached to a heteroaryl group, which is attached to the parent molecular moiety through an oxygen atom.

The term “LDL cholesterol” refers to low density lipoprotein.

The term “VLDL cholesterol” refers to very low density lipoprotein.

Non-toxic pharmaceutically acceptable salts include, but are not limited to salts of inorganic acids such as hydrochloric, sulfuric, phosphoric, diphosphoric, hydrobromic, and nitric or salts of organic acids such as formic, citric, malic, maleic, fumaric, tartaric, succinic, acetic, lactic, methanesulfonic, p-toluenesulfonic, 2-hydroxyethylsulfonic, salicylic and stearic. Similarly, pharmaceutically acceptable cations include, but are not limited to sodium, potassium, calcium, aluminum, lithium and ammonium. Those skilled in the art will recognize a wide variety of non-toxic pharmaceutically acceptable addition salts.

The invention also encompasses the prodrugs of the compounds of Formulas I and II. Those skilled in the art will recognize various synthetic methodologies that may be employed to prepare non-toxic pharmaceutically acceptable prodrugs of the compounds encompassed by Formulas I and II. Those skilled in the art will recognize a wide variety of non-toxic pharmaceutically acceptable solvents for preparing solvates, such as water, ethanol, mineral oil, vegetable oil, and dimethylsulfoxide.

The compounds of general Formulas I and II may be administered orally, topically, parenterally, by inhalation or spray or rectally in dosage unit formulations containing conventional non-toxic pharmaceutically acceptable carriers, adjuvants and vehicles. The term parenteral as used herein includes percutaneous, subcutaneous, intravascular (e.g., intravenous), intramuscular, or intrathecal injection or infusion techniques and the like. In addition, there is provided a pharmaceutical formulation comprising compounds of Formulas I and II and a pharmaceutically acceptable carrier. One or more compounds of general Formulas I and II may be present in association with one or more non-toxic pharmaceutically acceptable carriers and/or diluents and/or adjuvants, and if desired other active ingredients. The pharmaceutical compositions containing compounds of general Formulas I and II may be in a form suitable for oral use, for example, as tablets, troches, lozenges, aqueous or oily suspensions, dispersible powders or granules, emulsion, hard or soft capsules, or syrups or elixirs.

Compositions intended for oral use may be prepared according to any method known to the art for the manufacture of pharmaceutical compositions and such compositions may contain one or more agents selected from the group consisting of sweetening agents, flavoring agents, coloring agents and preservative agents in order to provide pharmaceutically elegant and palatable preparations. Tablets contain the active ingredient in admixture with non-toxic pharmaceutically acceptable excipients that are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques. In some cases such coatings may be prepared by known techniques to delay disintegration and absorption in the gastrointestinal tract and thereby provide a sustained action over a longer period. For example, a time delay material such as glyceryl monosterate or glyceryl distearate may be employed.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

Aqueous suspensions contain the active materials in admixture with excipients suitable for the manufacture of aqueous suspensions. Such excipients are suspending agents, for example sodium carboxymethylcellulose, methylcellulose, hydropropyl-methylcellulose, sodium alginate, polyvinylpyrrolidone, gum tragacanth and gum acacia; dispersing or wetting agents may be a naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose or saccharin.

Oily suspensions may be formulated by suspending the active ingredients in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents and flavoring agents may be added to provide palatable oral preparations. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents or suspending agents are exemplified by those already mentioned above. Additional excipients, for example sweetening, flavoring and coloring agents, may also be present.

Pharmaceutical compositions of the invention may also be in the form of oil-in-water emulsions. The oily phase may be a vegetable oil or a mineral oil or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol, anhydrides, for example sorbitan monooleate, and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol, glucose or sucrose. Such formulations may also contain a demulcent, a preservative and flavoring and coloring agents. The pharmaceutical compositions may be in the form of a sterile injectable aqueous or oleaginous suspension. This suspension may be formulated according to the known art using those suitable dispersing or wetting agents and suspending agents that have been mentioned above. The sterile injectable preparation may also be a sterile injectable solution or suspension in a non-toxic parentally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono-or diglycerides. In addition, fatty acids such as oleic acid find use in the preparation of injectables.

The compounds of general Formulas I and II may also be administered in the form of suppositories, e.g., for rectal administration of the drug. These compositions can be prepared by mixing the drug with a suitable non-irritating excipient that is solid at ordinary temperatures but liquid at the rectal temperature and will therefore melt in the rectum to release the drug. Such materials include cocoa butter and polyethylene glycols.

Compounds of general Formulas I and II may be administered parenterally in a sterile medium. The drug, depending on the vehicle and concentration used, can either be suspended or dissolved in the vehicle. Advantageously, adjuvants such as local anesthetics, preservatives and buffering agents can be dissolved in the vehicle.

In addition to pharmaceutical compositions, the compounds of Formulas I and II may be formulated as nutritional supplements or added to food so that a mammalian patient will take in the compounds with a meal. Such supplements and food products will be formulated or prepared so that the mammal, preferably a human, will take in an appropriate and effective amount of the compound with its diet.

Dosage levels of the order of from about 0.1 mg to about 140 mg per kilogram of body weight per day are useful in the treatment of the above-indicated conditions (about 0.5 mg to about 7 g per patient per day). The amount of active ingredient that may be combined with the carrier materials to produce a single dosage form will vary depending upon the host treated and the particular mode of administration. Dosage unit forms will generally contain between from about 1 mg to about 500 mg of an active ingredient.

It will be understood, however, that the specific dose level for any particular patient will depend upon a variety of factors including the activity of the specific compound employed, the age, body weight, general health, sex, diet, time of administration, route of administration, and rate of excretion, drug combination and the severity of the particular disease undergoing therapy.

For administration to non-human animals, the composition may also be added to the animal feed or drinking water. It may be convenient to formulate the animal feed and drinking water compositions so that the animal takes in a therapeutically appropriate quantity of the composition along with its diet. It may also be convenient to present the composition as a premix for addition to the feed or drinking water.

The disclosures in this application of all articles and references, including patents, are incorporated herein by reference.

The invention is illustrated further by the following examples, which are not to be construed as limiting the invention in scope or spirit to the specific procedures described in them.

The starting materials and various intermediates may be obtained from commercial sources, prepared from commercially available organic compounds, or prepared using well-known synthetic methods.

Representative examples of methods for preparing intermediates of the invention are set forth below.

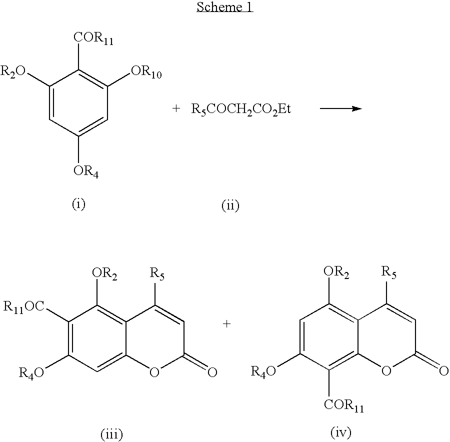

The coumarin derivates of the instant invention can be prepared according to the procedure described by Crombie et al., in Tet. Lett. 1985, 26(24), 2929-2932, as long as at least one of R₂, R₄, or R₁₀ is hydrogen. The known, commercially available acyl phloroglucinol (i) is reacted with a beta keto ester (ii) to form the isomeric compounds (iii) and (iv), which can be separated using standard chemical means including fractional recrystallization or chromatography.

The variables in the above structures carry the following definitions.

In Scheme 1, R₂ and R₄ carry the same definitions as set forth above with respect to Formulas I and II.

R₁₀ independently carries the same definition as R₂ and R₄ with the proviso that at least one of R₂, R₄, or R₁₀ is hydrogen.

R₁₁ is hydrogen or R₁₁ is an alkyl, alkenyl, alkynyl, arylalkyl, hydroxyalkyl, or —O-heteroarylalkyl group, where each group is unsubstituted or substituted with 1, 2, or 3 groups that are independently alkyl, alkoxy, halogen, CF₃, OCF₃ or nitro.

R₅ is hydrogen or R₅ is an alkyl, alkenyl, cycloalkyl, cycloalkylalkyl, aryl, arylalkyl, alkoxyalkyl, heteroaryl, heteroarylalkyl heterocycloalkyl, or heterocycloalkylalkyl group wherein each group is unsubstituted or substituted with 1, 2, or 3 groups that are independently selected from alkyl, alkoxy, halogen, CF₃, OCF₃, nitro, amino, mono or dialkylamino, carboxamido, or mono or dialkylcarboxamido.

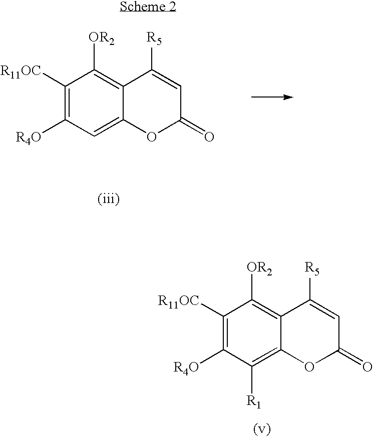

In Scheme 2, compound (iii) is alkylated or acylated using methods well known in the art to form compound (v).

R₁ is hydrogen, alkyl, alkenyl, alkanoyl, —O-alkanoyl, arylalkanoyl, —O-arylalkanoyl, heteroarylalkanoyl, —O-heteroarylalkanoyl, or hydroxyalkyl, wherein each group is unsubstituted or substituted with 1, 2, or 3 groups that are independently selected from alkyl, alkoxy, halogen, CF₃, OCF₃ or nitro;

R₂ and R₄ carry the same definitions as above;

R₁₁ is hydrogen or R₁₁ is an alkyl, alkenyl, alkynyl, arylalkyl, hydroxyalkyl, or —O-heteroarylalkyl group,

wherein each group is unsubstituted or substituted with 1, 2, or 3 groups that are independently alkyl, alkoxy, halogen, CF₃, OCF₃ or nitro.

R₅ is hydrogen or R₅ is an alkyl, alkenyl, cycloalkyl, cycloalkylalkyl, aryl, arylalkyl, alkoxyalkyl, heteroaryl, heteroarylalkyl heterocycloalkyl, or heterocycloalkylalkyl,

wherein each group is unsubstituted or substituted with 1, 2, or 3 groups that are independently alkyl, alkoxy, halogen, CF₃, OCF₃, nitro, amino, mono or dialkylamino, carboxamido, or mono or dialkylcarboxamido.

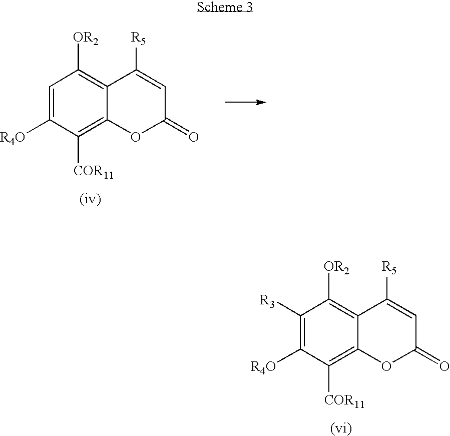

In Scheme 3, compound (iv) is alkylated or acylated using in scheme 3, methods well known in the art to form compound (vi.)

R₃ is selected from hydrogen or R₃ is an alkyl, alkenyl, alkanoyl, —O-alkanoyl, arylalkanoyl, —O-arylalkanoyl, heteroarylalkanoyl, —O-heteroarylalkanoyl, or hydroxyalkyl group,

wherein each group is unsubstituted or substituted with 1, 2, or 3 groups that are independently alkyl, alkoxy, halogen, CF₃, OCF₃ or nitro;

R₂ and R₄ carry the same definitions as above;

R₁₁ is hydrogen or R₁₁ is an alkyl, alkenyl, alkynyl, arylalkyl, hydroxyalkyl, or —O-heteroarylalkanyl group,

wherein each group is unsubstituted or substituted with 1, 2, or 3 groups that are independently alkyl, alkoxy, halogen, CF₃, OCF₃ or nitro.

R₅ is hydrogen or R₅ is an alkyl, alkenyl, cycloalkyl, cycloalkylalkyl, aryl, arylalkyl, alkoxyalkyl, heteroaryl, heteroarylalkyl, heterocycloalkyl, or heterocycloalkylalkyl group,

wherein each group is unsubstituted or substituted with 1, 2, or 3 groups that are independently alkyl, alkoxy, halogen, CF₃, OCF₃, nitro, amino, mono or dialkylamino, carboxamido, or mono or dialkylcarboxamido.

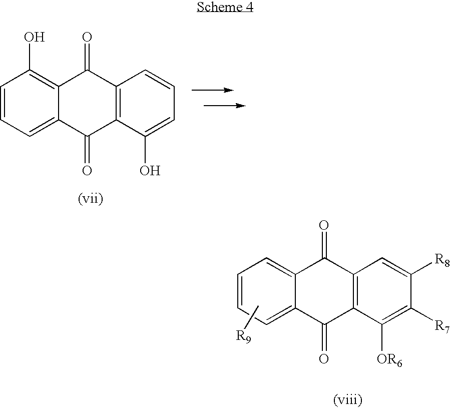

As shown in Scheme 4, the anthraquinone compounds of the instant invention are prepared from the commercially available compound (vii), which is commonly called anthrarufin or 1,5-dihydroxyanthraquinone, by methods well known in the art. See, for example, Bercich, et al., Aust. J. Chem. 1999, 52, 241-257; Bercich, et al., Aust. J. Chem. 1999, 52, 303-316; Cambie, et al., Aust. J. Chem. 1999, 52, 781-800; Bercich, et al., Aust. J. Chem. 1999, 52, 851-859.

In Scheme 4,

R₆ is hydrogen or an alkyl, alkoxyalkyl, alkanoyl, aryl, arylalkanoyl, or heteroarylalkanoyl group,

wherein each group is unsubstituted or substituted with 1, 2, or 3 groups that are independently alkyl, alkoxy, halogen, CF₃, OCF₃ or diazabicyclo[2.2.2]octyl;

R₇ and R₈ are independently alkyl, alkenyl, alkoxy, cycloalkyl, cycloalkylalkoxy, heterocycloalkyl, —CO₂H, —CO₂R₁₁,

wherein each of the above is optionally substituted with 1, 2, or 3 groups that are independently halogen, alkoxy, amino, diazabicyclo[2.2.2]octyl, or mono or dialkylamino;

wherein R₁₁ is alkyl, arylalkyl, aryl, or heterocycloalkylalkyl,

wherein each R₁₁ is optionally substituted with halogen, alkyl, alkoxy, hydroxy, CF₃, OCF₃, nitro, or diazabicyclo[2.2.2]octyl;

R₉ is selected from hydrogen or an alkyl, alkoxy, halogen, CF₃, OCF₃, amino, mono or dialkylamino, carboxamido, or mono or dialkylcarboxamido group,

wherein each alkyl group is optionally substituted with 1, 2, or 3 groups that are independently halogen, alkoxy, amino, diazabicyclo[2.2.2]octyl, or mono or dialkylamino.

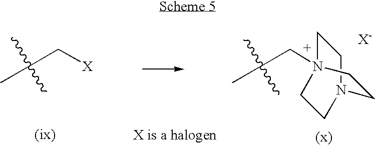

In scheme 5, compound (ix), which can be any of the above compounds as long as it contains an alkylhalide, is aminated using methods well known in the art. For example, see U.S. Pat. No. 5,994,391, column 323 for representative methodology. Although diazabicyclo[2.2.2]octyl is shown in scheme 5, other amines will also displace the halogen atom to form a carbon-nitrogen bond as exemplified by compound (x).

In Scheme 5, X is a leaving group, including halogen, methanesulfonate, trifluoromethanesulfonate, tosylate, brosylate, and nosylate.

Alternatively, the compounds of the instant application can be obtained by isolating them individually or as a mixture using methods well known in the art. Although a representative method is described below, other methods of extracting the compounds of interest from Mammea Americana, and Mammea Africana have been described in the scientific literature. Representative procedures are described in Carpenter, et al., J. Chem. Soc. (C) 1971, 3874; and Crombie, et al., J. Chem. Soc. (C) 1967, 2545.

Method of Extracting the Compounds of Interest:

M. Americana (dried fruit) samples are sequentially extracted with dichloromethane (DCM) and 95% ethanol. The organic extracts are evaporated to dryness under nitrogen flow and resuspended in dimethylsulfoxide (concentration of 20 mg/ml). Organic and water extracts are tested for ASBT inhibitory activity. The most active compounds are recovered from the DCM extract. The DCM extract is fractionated by reverse phase chromatography utilizing a C8 Luna column (4.6 mm×25 cm, 5μ particle size, 1 ml flow rate) and gradient elution. The gradient used for fractionation is depicted below:

Solvent A—acetonitrile (ACN):MeOH (1:1) with 0.1% HCOOH

Solvent B—Water with 0.1% HCOOH

Gradient Time Table

| Time | % A | % B | Flow |
|---|---|---|---|
| 0 | 75 | 25 | 1 ml |
| 20 | 100 | 0 | 1 ml |
| 32 | 100 | 0 | 1 ml |
| 35 | 75 | 25 | 1 ml |
| 45 | 75 | 25 | 1 ml |

The first 32 fractions were collected and tested for ASBT inhibitory activity in a taurocholate uptake assay. Fractions 14-16, showed significant inhibition of ASBT activity and consequently were individually rechromatographed using conditions similar to those described above, except that ACN:MeOH. (1:1) with 0.1% HCOOH was replaced with only ACN with 0.1% HCOOH. The chromatographic separation of fraction #15 produced a single fraction that contained all of the ASBT activity. The molecular ion (based on mass spectrometry) for the active component in this fraction was at m/z 406.

The leaf extract of Mammea Americana dose dependently inhibited bile acid transport (ASBT) activity with a 93% inhibition when used at 1% in the assay mixture (see FIG. 2).

The fruit extract of Mammea Americana was tested for ASBT inhibitory, 0.05 μl Of the fruit extract in the assay inhibited ASBT activity by more than 50%. When tested for specificity, the fruit extract was at least 50 times more specific for taurocholate (a bile acid) than for alanine uptake.

In summary, the fruit extract of Mammea Americana showed more than 50% inhibition of bile acid transport at 1 to 2000 dilution without cytotoxicity and is highly specific to taurocholate uptake. Accordingly, compounds of Formulas I and II have bile acid reabsorption inhibitory activity in vivo and are beneficial for reducing blood cholesterol levels and treating and preventing cardiovascular disease.

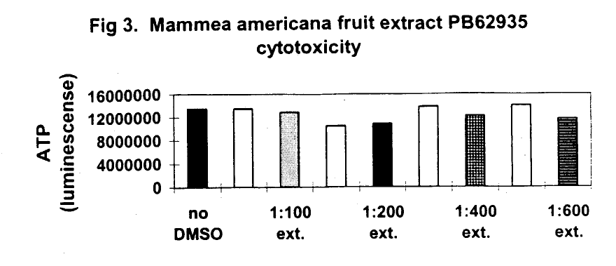

Cytotoxicity of the Mammea Americana fruit extract was determined in a cell viability assay by measuring the amount of cellular ATP (adenosine triphosphate) content. As shown in FIG. 3., cells were viable at up to maximum tested dose of 1% in the assay.

The invention and the manner and process of making and using it, are now described in such full, clear, concise and exact terms as to enable any person skilled in the art to which it pertains, to make and use the same. It is to be understood that the foregoing describes preferred embodiments of the present invention and that modifications may be made therein without departing from the spirit or scope of the present invention as set forth in the claims. To particularly point out and distinctly claim the subject matter regarded as invention, the following claims conclude this specification.

What is claimed is:

1. A method of inhibiting ileal bile acid transport protein comprising administering an effective amount of a compound of formula (I) and/or (II) or a pharmaceutically acceptable salt of I and/or II to a mammal 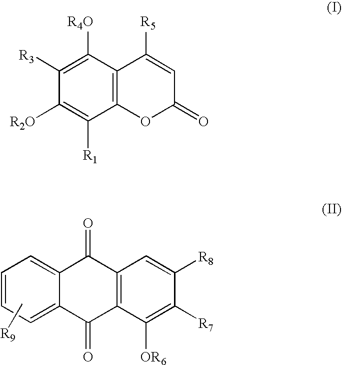
or their pharmaceutically acceptable salts,
wherein
R₁ and R₃ are independently hydrogen, alkyl, alkenyl, alkanoyl, —O-alkanoyl, arylalkanoyl, —O-arylalkanoyl, heteroarylalkanoyl, —O-heteroarylalkanoyl, or hydroxyalkyl,
wherein each group is unsubstituted or substituted with 1, 2, or 3 groups that are independently alkyl, alkoxy, halogen, haloalkyl, haloalkoxy, nitro or diazabicyclo[2.2.2]octyl;
R₂, R₄, and R₆ are independently hydrogen, alkyl, alkoxyalkyl, alkanoyl, aryl, arylalkanoyl, or heteroarylalkanoyl,
wherein each group is unsubstituted or substituted with 1, 2, or 3 groups that are independently alkyl, alkoxy, halogen, haloalkyl, haloalkoxy, or diazabicyclo[2.2.2]octyl;
R₅ is hydrogen, alkyl, alkenyl, cycloalkyl, cycloalkylalkyl, aryl, arylalkyl, alkoxyalkyl, heteroaryl, heteroarylalkyl heterocycloalkyl, or heterocycloalkylalkyl,
wherein each group is unsubstituted or substituted with 1, 2, or 3 groups that are independently alkyl, alkoxy, halogen, haloalkyl, haloalkoxy, nitro, amino, diazabicyclo[2.2.2]octyl, mono or dialkylamino, carboxamido, or mono or dialkylcarboxamido;
R₇ and R₈ are independently alkyl, alkenyl, alkoxy, cycloalkyl, cycloalkylalkoxy, heterocycloalkyl, —CO₂H, —CO₂R₁₁,
wherein each of the above is optionally substituted with 1, 2, or 3 groups that are independently halogen, alkoxy, amino, diazabicyclo[2.2.2]octyl, or mono or dialkylamino;
wherein R₁₁ is alkyl, arylalkyl, aryl, or heterocycloalkylalkyl,
wherein each R₁₁ is optionally substituted with halogen, alkyl, alkoxy, hydroxy, haloalkyl, haloalkoxy, nitro, or diazabicyclo[2.2.2]octyl;
R₉ is selected from hydrogen, alkyl, alkoxy, halogen, haloalkyl, haloakoxy, amino, mono or dialkylamino, carboxamido, or mono or dialkylcarboxamido,
wherein each alkyl group is optionally substituted with 1, 2, or 3 groups that are independently halogen, alkoxy, amino, diazabicyclo[2.2.2]octyl, or mono or dialkylamino.

2. A method according to claim 1 wherein R₅ is phenyl.

3. A method according to claim 2 wherein R₂ and R₄ are independently hydrogen, C₁-C₈ alkyl or alkanoyl.

4. A method according to claim 3 wherein R₁ and R₃ are independently hydrogen, C₁-C₈ alkyl or alkanoyl.

5. A method according to claim 1 wherein R₉ is hydrogen.

6. A method according to claim 5 wherein R₇ is alkyl, alkenyl or alkanoyl.

7. A method according to claim 6 wherein R₈ is hydrogen, alkyl, alkoxy or heterocycloalkyl, each of which is optionally substituted with up to four groups independently selected from C₁-C₆ alkyl, C₁-C₆ alkoxy, and halogen.

8. A method according to claim 1 wherein compound (II) is absent.

9. A method according to claim 1 wherein compound (I) is absent.

10. A method according to claim 1 directed to a method of inhibiting the activity of ileal apical sodium co-dependent bile acid transport protein comprising administering an effective amount of at least one of:
5,7-Dihydroxy-8-(3-methyl-but-2-enyl)-6-(2-methyl-butyryl)-4-phenyl-chromen-2-one;
5,7-Dihydroxy-6-(3-methyl-but-2-enyl)-8-(3-methyl-butyryl)-4-phenyl-chromen-2-one;
5,7-Dihydroxy-6-(3-methyl-but-2-enyl)-8-(2-methyl-butyryl)-4-phenyl-chromen-2-one;
5,7-Dihydroxy-8-(3-methyl-but-2-enyl)-6-(3-methyl-butyryl)-4-phenyl-chromen-2-one; or
1-Hydroxy-2-(2-methyl-allyl)-3-(4,4,6-trimethyl-[1,3]dioxan-2-yl)-anthraquinone.

11. A method of lowering blood cholesterol levels comprising administering an effective amount of a compound of formula (I) and/or formula (II) or a pharmaceutically acceptable salt of I and/or II to a mammal: 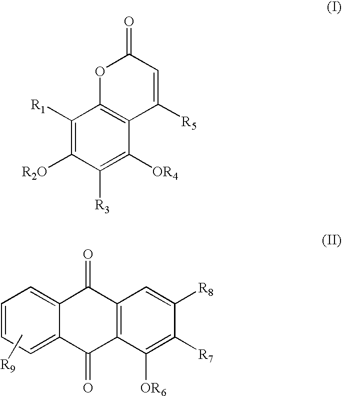
wherein
R₁ and R₃ are independently hydrogen, alkyl, alkenyl, alkanoyl, —O-alkanoyl, arylalkanoyl, —O-arylalkanoyl, heteroarylalkanoyl, —O-heteroarylalkanoyl, or hydroxyalkyl,
wherein each group is unsubstituted or substituted with 1, 2, or 3 groups that are independently alkyl, alkoxy, halogen, haloakyl, haloalkoxy, nitro or diazabicyclo[2.2.2]octyl;
R₂, R₄, and R₆ are independently hydrogen, alkyl, alkoxyalkyl, alkanoyl, aryl, arylalkanoyl, or heteroarylalkanoyl,
wherein each group is unsubstituted or substituted with 1, 2, or 3 groups that are independently alkyl, alkoxy, halogen, haloalkyl, haloalkoxy, or diazabicyclo[2.2.2]octyl;
R₅ is hydrogen, alkyl, alkenyl, cycloalkyl, cycloalkylalkyl, aryl, arylalkyl, alkoxyalkyl, heteroaryl, heteroarylalkyl heterocycloalkyl, or heterocycloalkylalkyl,
wherein each group is unsubstituted or substituted with 1, 2, or 3 groups that are independently alkyl, alkoxy, halogen, haloalkyl, haloalkoxy, nitro, amino, diazabicyclo[2.2.2]octyl, mono or dialkylamino, carboxamido, or mono or dialkylcarboxamido;
R₇ and R₈ are independently alkyl, alkenyl, alkoxy, cycloalkyl, cycloalkylalkoxy, heterocycloalkyl, —CO₂H, —CO₂R₁₁,
wherein each of the above is optionally substituted with 1, 2, or 3 groups that are independently halogen, alkoxy, amino, diazabicyclo[2.2.2]octyl, or mono or dialkylamino;
wherein R₁₁ is alkyl, arylalkyl, aryl, or heterocycloalkylalkyl,
wherein each R₁₁ is optionally substituted with halogen, alkyl, alkoxy, hydroxy, haloalkyl, haloalkoxy, nitro, or diazabicyclo[2.2.2]octyl;
R₉ is selected from hydrogen, alkyl, alkoxy, halogen, haloalkyl, haloalkoxy, amino, mono or dialkylamino, carboxamido, or mono or dialkylcarboxamido,
wherein each alkyl group is optionally substituted with 1, 2, or 3 groups that are independently halogen, alkoxy, amino, diazabicyclo[2.2.2]octyl, or mono or dialkylamino.

12. A method according to claim 11 wherein R₅ is phenyl.

13. A method according to claim 12 wherein R₂ and R₄ are independently hydrogen, C₁-C₈ alkyl or alkanoyl.

14. A method according to claim 13 wherein R₁ and R₃ are independently hydrogen, C₁-C₈ alkyl or alkanoyl.

15. A method according to claim 11 wherein R₉ is hydrogen.

16. A method according to claim 15 wherein R₇ is alkyl, alkenyl or alkanoyl.

17. A method according to claim 16 wherein R₈ is hydrogen, alkyl, alkoxy or heterocycloalkyl, each of which is optionally substituted with up to four groups independently selected from C₁-C₆ alkyl, C₁-C₆ alkoxy, and halogen.

18. A method according to claim 11 wherein compound (II) is absent.

19. A method according to claim 11 wherein compound (I) is absent.

20. A method according to claim 11 of lowering blood cholesterol levels in a mammal comprising administering an effective amount of at least one of:
5,7-Dihydroxy-8-(3-methyl-but-2-enyl)-6-(2-methyl-butyryl)-4-phenyl-chromen-2-one;
5,7-Dihydroxy-6-(3-methyl-but-2-enyl)-8-(3-methyl-butyryl)-4-phenyl-chromen-2-one;
5,7-Dihydroxy-6-(3-methyl-but-2-enyl)-8-(2-methyl-butyryl)-4-phenyl-chromen-2-one;
5,7-Dihydroxy-8-(3-methyl-but-2-enyl)-6-(3-methyl-butyryl)-4-phenyl-chromen-2-one; or
1-Hydroxy-2-(2-methyl-allyl)-3-(4,4,6-trimethyl-[1,3]dioxan-2-yl)-anthraquinone.

21. A pharmaceutical composition containing an effective amount of a compound of formula (I) and/or (II) or a pharmaceutically acceptable salt of I or II: 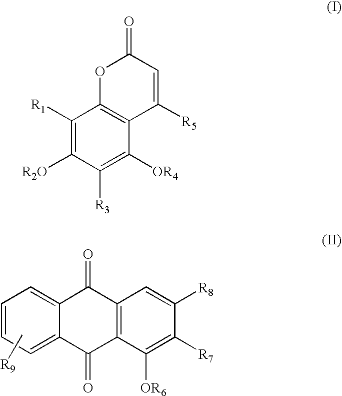
wherein
R₁ and R₃ are independently hydrogen, alkyl, alkenyl, alkanoyl, —O-alkanoyl, arylalkanoyl, —O-arylalkanoyl, heteroarylalkanoyl, —O-heteroarylalkanoyl, or hydroxyalkyl,
wherein each group is unsubstituted or substituted with 1, 2, or 3 groups that are independently alkyl, alkoxy, halogen, haloalkyl, haloalkoxy, nitro or diazabicyclo[2.2.2]octyl;
R₂, R₄, and R₆ are independently hydrogen, alkyl, alkoxyalkyl, alkanoyl, aryl, arylalkanoyl, or heteroarylalkanoyl,
wherein each group is unsubstituted or substituted with 1, 2, or 3 groups that are independently alkyl, alkoxy, halogen, haloalkyl, haloalkoxy, or diazabicyclo[2.2.2]octyl;
R₅ is hydrogen, alkyl, alkenyl, cycloalkyl, cycloalkylalkyl, aryl, arylalkyl, alkoxyalkyl, heteroaryl, heteroarylalkyl heterocycloalkyl, or heterocycloalkylalkyl,
wherein each group is unsubstituted or substituted with 1, 2, or 3 groups that are independently alkyl, alkoxy, halogen, haloalkyl, haloalkoxy, nitro, amino, diazabicyclo[2.2.2]octyl, mono or dialkylamino, carboxamido, or mono or dialkylcarboxamido;
R₇ and R₈ are independently alkyl, alkenyl, alkoxy, cycloalkyl, cycloalkylalkoxy, heterocycloalkyl, —CO₂H, —CO₂R₁₁,
wherein each of the above is optionally substituted with 1, 2, or 3 groups that are independently halogen, alkoxy, amino, diazabicyclo[2.2.2]octyl, or mono or dialkylamino;
wherein R₁₁ is alkyl, arylalkyl, aryl, or heterocycloalkylalkyl,
wherein each R₁₁ is optionally substituted with halogen, alkyl, alkoxy, hydroxy, haloalkyl, haloalkoxy, nitro, or diazabicyclo[2.2.2]octyl;
R₉ is selected from hydrogen, alkyl, alkoxy, halogen, haloalkyl, haloalkoxy, amino, mono or dialkylamino, carboxamido, or mono or dialkylcarboxamido,
wherein each alkyl group is optionally substituted with 1, 2, or 3 groups that are independently halogen, alkoxy, amino, diazabicyclo[2.2.2]octyl, or mono or dialkylamino;
and at least one pharmaceutically acceptable carrier, adjuvant or excipient.

22. A composition according to claim 21 wherein R₅ is phenyl.

23. A composition according to claim 22 wherein R₂ and R₄ are independently hydrogen, C₁-C₈ alkyl or alkanoyl.

24. A composition according to claim 23 wherein R₁ and R₃ are independently hydrogen, C₁-C₈ alkyl or alkanoyl.

25. A composition according to claim 21 wherein R₉ is hydrogen.

26. A composition according to claim 25 wherein R₇ is alkyl, alkenyl or alkanoyl.

27. A composition according to claim 26 wherein R₈ is hydrogen, alkyl, alkoxy or heterocycloalkyl, each of which is optionally substituted with up to four groups independently selected from C₁-C₆ alkyl, C₁-C₆ alkoxy, and halogen.

28. A composition according to claim 21 wherein compound (II) is absent.

29. A composition according to claim 21 wherein compound (I) is absent.

30. A pharmaceutical composition according to claim 21 containing an effective amount of at least one of:
5,7-Dihydroxy-8-(3-methyl-but-2-enyl)-6-(2-methyl-butyryl)-4-phenyl-chromen-2-one;
5,7-Dihydroxy-6-(3-methyl-but-2-enyl)-8-(3-methyl-butyryl)-4-phenyl-chromen-2-one;
5,7-Dihydroxy-6-(3-methyl-but-2-enyl)-8-(2-methyl-butyryl)-4-phenyl-chromen-2-one;
5,7-Dihydroxy-8-(3-methyl-but-2-enyl)-6-(3-methyl-butyryl)-4-phenyl-chromen-2-one; or
1-Hydroxy-2-(2-methyl-allyl)-3-(4,4,6-trimethyl-[1,3]dioxan-2-yl)-anthraquinone; and a pharmaceutically acceptable carrier, adjuvant or excipient.

31. The use of a pharmaceutical composition according to claim 21 for the manufacture of a medicament for inhibiting ileal apical sodium co-dependent bile acid transport protein.

32. A packaged pharmaceutical composition comprising the pharmaceutical composition of claim 21 in a container and instructions for using the composition to inhibit ileal apical sodium co-dependent bile acid transport protein.

33. The use of a pharmaceutical composition according to claim 21 for the manufacture of a medicament for the reduction of blood cholesterol levels.

34. A packaged pharmaceutical composition comprising the pharmaceutical composition of claim 21 in a container and instructions for using the composition to reduce blood cholesterol levels.
